(19) United States
(12) Patent Application Publication
(10) Pub. No.: US 2024/0398319 A1
(43) Pub. Date: Dec. 5, 2024

(54) Method for selecting a portion of an encephalographic signal, devices and corresponding program

(71) Applicants: CENTRE NATIONAL DE LA RECHERCHE SCIENTIFIQUE, Paris (FR); UNIVERSITE TOULOUSE III – PAUL SABATIER, Toulouse Cedex 9 (FR); ECOLE NATIONALE DE L’AVIATION CIVILE, Toulouse (FR)

(72) Inventors: Ludovic Gardy, Toulouse Cedex (FR); Emmanuel Barbeau, Toulouse Cedex (FR); Christophe Hurter, Toulouse Cedex (FR)

(21) Appl. No.: 18/701,778

(22) PCT Filed: Oct. 20, 2022
(86) PCT No.: PCT/EP2022/079304

§ 371 Date: Apr. 16, 2024

(30) Foreign Application Priority Data
Oct. 20, 2021 (FR) 2111138

(51) Int. Cl. A61B 5/372; A61B 5/00

(52) U.S. Cl. CPC A61B 5/372; A61B 5/726; A61B 5/7264

(57) ABSTRACT

A method for selecting data derived from an electroencephalogram, in the form of a set of starting scalograms, each scalogram being calculated from a portion of an electroencephalographic signal. The method includes: extracting, via an artificial neural network, a set of candidate scalograms; and for some candidate scalograms of the set of candidate scalograms: calculating characteristics of the electroencephalographic signal portion corresponding to the candidate scalogram; and when the plurality of characteristics are within prerequisite value ranges, selecting the electroencephalographic signal portion of the candidate scalogram within an electroencephalographic signal selection data structure.

### TECHNICAL FIELD

The invention relates to the field of electroencephalographic data processing. More particularly, the invention relates to the processing of data derived from electroencephalograms originating from patients in order to facilitate research of anomalies within these electroencephalograms. Still more particularly, the invention relates to the identification, detection and/or classification, within the electroencephalographic signals, of a signal portion comprising potential anomalies.

### PRIOR ART

From many years, the electroencephalogram is a favourite tool for practitioners in the search and identification of neurological pathologies, like for example the Parkinson's disease, the Alzheimer's disease or epilepsy. The advantage of the electroencephalogram is that it allows having signals representative of the electroencephalographic activity which can be analysed and studied in order to detect anomalies. Traditionally, the signals may be obtained either by means of electrodes placed on the scalp of the patient (using an electrode helmet) or from electrodes implanted beneath the surface of the skull, either at the surface of or deep in the cerebral tissue (it is then referred to as intracranial electroencephalography-iEEG). Thus, for example, the study of epilepsies poses several challenges to professionals because it could take on several forms and involves complex spatio-temporal and multi-scale physio-pathological processes. Thus, in order to locate the diseased cerebral zones and to proceed with the possible surgical exeresis thereof, patients suffering from drug-resistant epilepsy sometimes have to be hospitalised for several consecutive weeks. Their electroencephalographic activity is recorded by means of deep electrodes, implanted directly inside the cerebral structures, in order to identify one or more epileptogenic zone(s) (ZE). These recordings comprise periods of rest and periods of seizures which are recorded to study the emergence, the dynamics and the semiology thereof. The amount and the heterogeneity of the data that are gathered during hospitalisation pose a major challenge to practitioners to establish a diagnosis. To date, there is no automated tool for assisting them in carrying out analyses that are sometimes very long and difficult, in particular on electroencephalography (EEG) data, the volumes of which are very large. Indeed, during the hospitalisation period, one objective is to record one or more seizure(s) (critical period) in the patient, preferably spontaneous, in order to characterise the epileptogenic network. It is also possible to search for electrophysiological markers of the network during intercritical periods, i.e. outside the seizures. The investigation of these markers is primarily carried out manually, by the practitioner, through visual explorations of the cerebral activity. These markers are demonstrators of the epileptogenic activity of local regions or of wider networks. They are intended to guide clinical hypotheses by delimiting the epileptogenic and propagation zones. One could differentiate two types of main intercritical markers: the intercritical points (PEIs) and the high-frequency oscillations (HFOs) and in particular Fast Ripples (“Fast Ripple”-FR). Recent researches have highlighted a relationship between high-frequency oscillations (HFOs in general and FR in particular) appearing in an intercritical period and an epileptogenic network, and possibly an epileptogenic zone itself. The problem, for the practitioner, consists in identifying these high-frequency oscillations, in recordings over several consecutive hours. Indeed, a problem encountered with HFOs is that they take on the form of very low and local signals (i.e. located). The greater the scale, the larger the volumes and therefore the captured neural populations will be. Increasing the scale makes the HFOs likely to be drowned in the surrounding turmoil, until they become undetectable. Thus, to analyse 10 minutes of cerebral activity recorded over 10 channels, the practitioner needs about 10 hours. Besides being time-consuming, this activity is very laborious, difficult and subject to more or less significant cognitive biases from the practitioner, leading to substantial differences in detection between practitioners.

Thus, methods for automatically detecting anomalies, in particular HFOs, have been explored in recent years, in order to facilitate and objectivise this detection (i.e. by suppressing the cognitive biases of practitioners).

One method consists in an automatic analysis of the signal, through the use of the Dynamic Time Warping technique (in English, Dynamic Time Warping—DTW) allowing measuring the similarity between two sequences which could vary over time. This method is effective when it comes to recognise patterns that are barely variable in a relatively stable environment, but is dangerously failed as the shape of the investigated signal departs from the request. The PEIs are events that creates difficulty to the DTW because their spatio-temporal dynamics, therefore their morphology, vary depending on the patients and the cerebral zones where they are recorded, but also because they are located in EEG signals carrying numerous “false-friends”, which could look like them and fool the algorithm, like K-complexes or sleep spindles.

Other methods are based on machine-learning. The machine-learning methods have the advantage of determining alone the rules to be applied to decide that an event belongs to one category or another, like the PEI category if it has been trained over a sufficiently large number of examples.

The first-generation automatic detectors, based only on conventional techniques for processing the raw or filtered signal and the application of fixed or adaptive amplitude thresholds have proven that they could partially address the problem (Staba et al., 2002; Gardner et al., 2007; Crepon et al., 2010; Zelmann et al., 2012). Nevertheless, they are not competitive enough in comparison with a manual search process. The results obtained on simulated data are sometimes encouraging, but already insufficient, since the used simulations supply a quality signal, i.e. composed of relatively “clean” events and a stable baseline.

Machine-learning models have given rise to a new way of approaching the problem. The discriminating parameters of the FRs are sometimes difficult to objectivise: a practitioner knows that there is an FR on a signal portion without being necessarily capable of listing all of the elements that allows asserting it: he/she has learned to recognise them because they are eccentric with respect to a standard, integrated more or less implicitly. During a learning process, the model will also identify and integrate discriminating criteria that enables it to distinguish an event of interest like an FR, from other types of events. Few studies have also emerged for detecting FRs, or intercritical markers (HFOs) in general, based on machine-learning algorithms and even less on a large scale.

In particular, a MOSSDET detector (Lachner-Piza et al. 2020) has been proposed, it uses support vector machines (SVM in English). The MOSSDET performances are higher than those of the other detectors currently published. Yet, surprisingly, a MOSSDET detects events even with a signal-to-noise ratio equal to 0 dB, which poses a problem under real conditions and on a noisy signal, since it is difficult to actually consider these events the intensity or power of which does not exceed the background activity as HFOs or FRs. This excess sensitivity with a low signal-to-noise ratio is reflected by the detection of large amounts of false positives and is therefore problematic.

Other deep-learning based techniques have also been developed, but these are either based on very good quality datasets, or cause the emission of too many false positives, which does not facilitate the subsequent classification work, because these false positives result in an increase in the workload for the practitioner.

In conclusion, the automatic detection attempts that have been developed until now have encountered blocking difficulties to consider, on this date, a use thereof in clinical practice. Thus, it is desirable to provide an anomaly detection technique that could facilitate the processing of large amounts of data as explained before while limiting the detection of false positives, i.e. events of the signal that are not representative of the events to be detected.

### SUMMARY OF THE INVENTION

One objective of the invention is to enable an easier location of zones of interest within an electroencephalogram that is extended over time. More particularly, a method is provided for selecting data derived from an electroencephalogram, said data being in the form of a set of starting scalograms, each scalogram of the set of starting scalograms being calculated from a portion of an electroencephalographic signal acquired beforehand. Such a method comprises:
a step of extracting, from a set of starting scalograms, via an artificial neural network, a set of candidate scalograms;
   For at least some candidate scalograms of the set of candidate scalograms:
a step of calculating a plurality of characteristics of the electroencephalographic signal portion corresponding to the candidate scalogram; and
when the plurality of characteristics of the electroencephalographic signal portion of the candidate scalogram are within prerequisite value ranges, a step of selecting the electroencephalographic signal portion of the candidate scalogram within an electroencephalographic signal selection data structure.

Thus, unlike a simplistic method consisting in using a neural network to differentiate the characteristics of the signal, the invention implements a neural network on the scalograms before calculation of the characteristics on the candidate signal portions derived from the first selection by neural network. This allows calculating the characteristics only over a reduced sample of signal portions and counter-intuitively, being more efficient on the processing of these large data sets, and therefore more efficient in consumed resources. The applicability of the method described herein is not limited to epilepsy. More particularly, the method may be used for processing other types of electroencephalographic signals and pathologies other than epilepsy. The interest of the described method is that it mixes the deep-learning and signal processing approaches in order to result in a drastic decrease in false positives, which have limited the clinical use of the previously-described methods.

According to a particular feature, said artificial neural network is a convolutional neural network.

According to a particular feature, said artificial neural network is pre-trained to detect “fast ripple” type fast oscillations within the scalograms.

More particularly, in a specific embodiment, one objective is to locate high-frequency oscillations in a simple and efficient manner in order to allow for a significant improvement in the epilepsy diagnosis process, thanks to a faster and more accurate location of the tissues to be treated and/or to be operated.

According to a particular feature, the characteristics of the electroencephalographic signal portion that are calculated belong to the group comprising at least: its duration, its signal-to-noise ratio, the number of oscillations that compose the event, the amplitude of these oscillations, the shape of the oscillations.

According to a particular feature, the calculation of the scalograms of the set of starting scalograms comprises:
a step of segmenting an input electroencephalographic signal, according to a predetermined segmentation duration, outputting a plurality of electroencephalographic signal portions;
a step of spectral equalisation of each electroencephalographic signal portion, outputting a plurality of equalised electroencephalographic signal portions;
a step of calculating, from each equalised electroencephalographic signal portion, a scalogram using a wavelet transform;

According to a particular feature, the calculation of the scalograms of the set of starting scalograms further comprises, for each scalogram obtained using a wavelet transform, a step of normalising the scalogram.

According to a particular feature, the plurality of characteristics calculated within the electroencephalographic signal portion corresponding to the candidate scalogram comprises:
a step of calculating a Hilbert envelope of the electroencephalographic signal portion;
a step of selecting, within the Hilbert envelope, the set of points located beyond the 97.5th percentile, so-called extrema set;
a step of selecting the set of extreme points following one another, with no interruption greater than 2 ms and the total duration of which is at least equal to 6 ms; and
a step of calculating the amplitude and the number of positive peaks of the signal over the set of previously selected points.

According to a particular feature, the step of selecting the electroencephalographic signal portion of the candidate scalogram within an electroencephalographic signal selection data structure occurs when the average amplitude of the points of the set of previously selected points is at least twice as high as the amplitude of all of the other points of the Hilbert envelope and when at least four positive peaks are present on all of the previously selected points.

According to another aspect, the invention also relates to a device for selecting data derived from an electroencephalogram, said data being in the form of a set of starting scalograms, each scalogram of the set of starting scalograms being calculated from a portion of an electroencephalographic signal acquired beforehand, said device being characterised in that it comprises:
means for extracting, from the set of starting scalograms, via an artificial neural network [trained, configured], a set of candidate scalograms;
   For at least some candidate scalograms of the set of candidate scalograms:
means for calculating a plurality of characteristics of the electroencephalographic signal portion corresponding to the candidate scalogram; and
means for selecting the electroencephalographic signal portion of the candidate scalogram within an electroencephalographic signal selection data structure, said means being activated when the plurality of characteristics of the electroencephalographic signal portion of the candidate scalogram are within prerequisite value ranges.

The invention also relates to an information medium readable by a data processor, and including instructions of a program as mentioned hereinabove.

The information medium may be any entity or device capable of storing the program. For example, the support may include a storage means, such as a ROM, for example a CD ROM or a microelectronic circuit ROM, or a magnetic recording means, for example a floppy disk or a hard disk.

Besides, the information medium may be a transmissible medium such as an electrical or optical signal, which may be conveyed via an electrical or optical cable, by radio or by other means. In particular, the program according to the invention may be downloaded over an Internet-type network.

Alternatively, the information medium may be an integrated circuit in which the program is incorporated, the circuit being adapted to execute or to be used in the execution of the considered method.

According to one embodiment, the invention is implemented by means of software and/or hardware components. In this respect, the term “module” may correspond in this document to a software component, to a hardware component or to a set of hardware and software components.

A software component corresponds to one or more computer program(s), one or more sub-program(s) of a program, or more generally to any element of a program or software capable of implementing a function or a set of functions, according to what is described hereinbelow for the considered module. Such a software component is executed by a data processor of a physical entity (terminal, server, etc.) and is capable of accessing the hardware resources of this physical entity (memories, recording media, communication buses, input/output electronic boards, user interfaces, etc.).

Similarly, a hardware component corresponds to any element of a hardware set capable of implementing a function or a set of functions, according to what is described hereinbelow for the considered module. This may consist of a hardware component which is programmable or has an integrated processor for executing software, for example an integrated circuit, a smart card, a memory card, an electronic card for executing a firmware, etc.

### BRIEF DESCRIPTION OF THE DRAWINGS

Other features and advantages will appear more clearly upon reading the following description of a preferred embodiment, given as a simple illustrative and non-limiting example, and from the appended drawings, wherein:

FIG. 1 describes the general principle of the proposed technique;

FIG. 2 describes the principle of generation of the scalograms from electroencephalographic signals;

FIG. 3 is an illustration of several electroencephalographic events in the form of scalograms;

FIG. 4 is an illustration of the architecture of the convolutional neural network used for the implementation of the present technique in one embodiment;

FIG. 5 describes the simplified architecture of a data processing device.

### DETAILED DESCRIPTION

### Reminders of the Principle

As indicated before, to solve the problems currently encountered, in particular regarding the detection of false positives, the Inventors have had the idea of combining both deep-learning techniques, which allow obtaining a first recording set of EEG signals corresponding to a given number of criteria, and signal processing analytical techniques, implemented on this first recording set of EEG signals, to enable rejection of false positives. In other words, rather than attempting, obtaining via one single technique, a recording set comprising only effective points of interest, corresponding to all of the pre-established criteria for the detection of these points or of these areas of interest, the proposed technique operates in two complementary segregation steps: the first one, based on deep-learning, via a convolutional neural network for example, allows working on a large mass of input data, to keep only candidate signal portions. Then, in the second step, starting from the candidate signal portions, one or more signal analysis technique(s) is/are applied to determine whether this portion should be kept and tagged, or not, for a subsequent analysis. The final objective is to provide a practitioner (or another complementary analysis device) only with the candidate portions that are most likely to include characteristics of interest. This way of proceeding so is counter-intuitive, since a person skilled in the art would have rather a tendency to work first on the analysis of the signal, and then to supply the analysed portions to the neural network. This is not the approach considered by the Inventors, which approach consists in mass-processing the data via a neural network, then, once a pre-selection has been made by this neural network, analysing the candidate signal portions more finely.

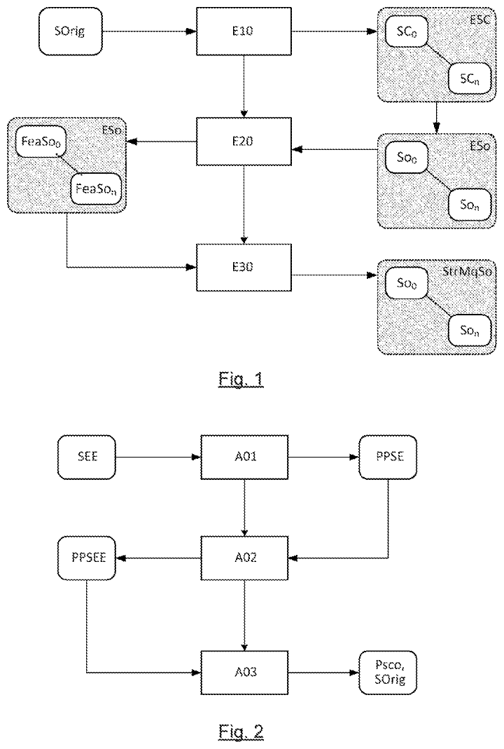

Thus, in general, with reference to FIG. 1, the technique relates to a method for selecting data derived from an electroencephalogram (EEG), said data being in the form of a set of starting scalograms, each scalogram of the set of starting scalograms being calculated from a portion of an electroencephalographic signal acquired beforehand, the method comprising:
a step (E10) of extracting, from the set of starting scalograms (SOrig), via an artificial neural network, a set of candidate scalograms (Esc);
   For at least some candidate scalograms (Sc) of the set of candidate scalograms (Esc):
a step (E20) of calculating a plurality of characteristics (FeaSo) of the electroencephalographic signal portion (PSo) corresponding to the candidate scalogram (Sc); and
when the plurality of characteristics of the electroencephalographic signal portion (So) of the candidate scalogram (Sc) are within prerequisite value ranges, a step (E30) of marking the electroencephalographic signal portion (So) of the candidate scalogram (Sc) within an electroencephalographic signal marking data structure (StrMqSo).

Depending on the implemented operating conditions, the electroencephalogram is intracranial, i.e. recorded from electrodes implanted in the cerebral cortex (stereo-electroencephalography (SEEG), which consists in implanting intracerebral electrodes to a patient, who remains hospitalised for about ten days, during which his/her intracerebral activity is continuously recorded, over more than one hundred recording channels distributed in the brain structures suspected of being involved in a brain pathology), or at the surface of the latter. In other situations, the electroencephalogram may be obtained using a conventional or high-density helmet (with a number of electrodes greater than 48). Thus, an ecological approach for pre-detection and/or classification of events of interest in two steps is provided intended to limit, for example, the subsequent visual and manual workload of the practitioner and/or the calculation load for a subsequent automated processing of the candidate event. The first step (E10) exploits the processing capacity of the images of a neural network trained to detect the trace of the events of interest (like for example HFOs, FRs, a non-sinusoidal shape of beta oscillations, increased power in the delta (2-4 Hz) and theta (4-8 Hz) frequency bands, a decrease in the power of the posterior alpha (8-12 Hz) and/or beta (13-30 Hz)—Parkinson—or other events in the case of Alzheimer's disease or other neurodegenerative diseases) on a set of scalograms made out of electroencephalogram signals. When a candidate event of interest is detected by the network, from the scalogram representative of the signal of this candidate event of interest, the second step (E20) is implemented to verify the result at the output of the network and eliminate false alarms (false positives). This second step involves different techniques for quantitative analyses of the portion of the original filtered signal having been used to create the candidate scalogram upon completion of step E10. Several characteristics of the candidate event of interest may be calculated: for example its duration, its signal-to-noise ratio or the number of oscillations that compose the event, the amplitude of these oscillations, the shape of the oscillations, etc. This method operates on large amounts of raw data, even noisy data, on the macro and micro scales. The multi-step, multi-scale dual nature of this method allows detecting many events of interest without a prohibitive number of false positives contaminates the results. An interpretation and/or classification (of the pre-selected data) may subsequently be carried out simply and quickly to characterise the zone of interest at the origin of these events of interest. Thus, the described method allows using artificial intelligence processes to process large amounts of data upstream, outputting a set of candidate events of interest, the events of this set being analysed afterwards, in order to limit the amount of potential false positives. Thus, the number of events that have actually to be analysed in order to confirm or deny the presence of an anomaly is considerably reduced.

Depending on the embodiments, and on the target objective, the calculation of the scalograms from the signals may be more or less complex. For example, some events, such as HFOs, are characterised by a signal-to-noise ratio (SNR) that is often relatively low. It could be necessary to reinforce the trace of these events in the “time-frequency” space and to apply a normalisation of the considered events. In general, the scalograms are obtained in the following manner, described with reference to FIG. 2, Thus, the calculation of the scalograms of the set of starting scalograms comprises:
a step (A01) of segmenting an input electroencephalographic signal (SEE), according to a predetermined segmentation duration (dSp), outputting a plurality of electroencephalographic signal portions (PPSE);
a step (A02) of spectral equalisation of each electroencephalographic signal portion of the PPSE, outputting a plurality of equalised electroencephalographic signal portions (PPSEE);
a step of calculating, from each equalised electroencephalographic signal portion, a scalogram using a wavelet transform, outputting a plurality of scalograms (PSco, SOrig).

Moreover, the creation of the scalograms also comprises a phase of associating, in the marking database, the original signal portions having been used as a basis for the creation of the scalograms, so that the obtainment of the original signal portion, from a candidate scalogram, is facilitated for the analytical processing of this signal portion.

In a general case, the scalograms are obtained from the segmented signal over a short period (for example comprised between 300 ms and 500 ms, typically 400 ms). Depending on the embodiments, the time windows for extracting the signal may be sliding, with a suitable overlap period (for example 50 ms). This overlap may be reduced or increased in particular according to the available computing resources, just like the period of the segmented signal.

Once segmented, the raw signal may be (and is often) pre-whitened. The pre-whitening, also called spectral equalisation, whitening or pre-emphasis, applied to the raw signal has the effect of suppressing the continuous component of the signal and the low frequencies. Several methods may be considered. However, the Inventors have decided to prefer the FOBaD (or Diff) method, standing for “first-order backward differencing”, which could be expressed according to the following equation where x is the pre-whitened signal and n the position of a value in the signal.

$$\overline{x} [ n ] = x [ n ] - x [ n - 1 ]$$

Initial scalograms are obtained after wavelet transformation of the pre-whitened signal x̅.

∫_{-∞}^{+∞}f(x̅)ψ_{(a,b)}*x̅dx

The obtained power spectral densities take into account the amplitude and the frequency of the oscillations in the signal.

Depending on the initial characteristics of the events to be detected, the initial scalograms (derived from the wavelet transform) could be normalised with the ZHO method having the effect of whitening the signal by equalising the frequencies composing the background noise, which then becomes similar to white noise. The more the signal of interest features a significant spectral power, the more its power is overestimated by the z-score and its trace reinforced, for an optimised SNR (N Roehri, 2016).

The ZHO parameter forces the real and imaginary coefficients to adopt a similar distribution throughout all frequencies. This normalisation technique can be applied only to short time windows. Otherwise, the parameters of the normalisation might undergo a bias that could cause a deterioration in performance. The advantage of this method is that it is adaptive and does not require the definition of any baseline, which is interesting in the case of the present technique.

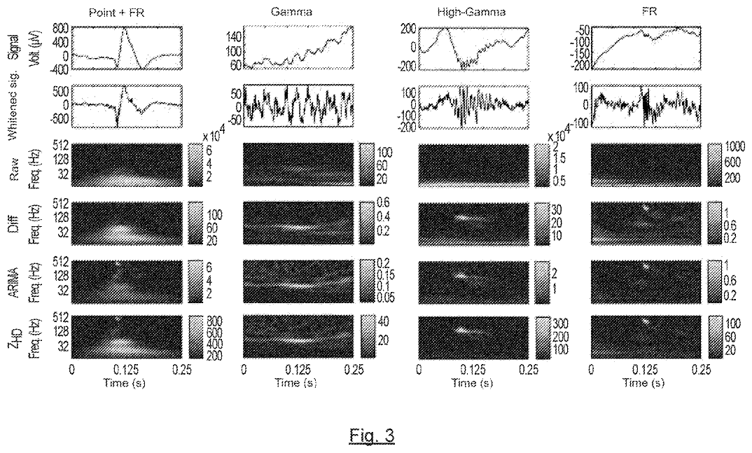

FIG. 3 illustrates the results of the processing applied to the signal portions in four different situations. More specifically, in FIG. 2, four 250 ms events in the time-frequency domain. From left to right, it is possible to observe a simultaneous intercritical point with a fast ripple, a gamma oscillation (<80 Hz), a high-frequency gamma oscillation (HG, 80-150 Hz) and a fast ripple alone (>200 Hz). The first line represents the raw signal, the second pre-whitened signal. The third line illustrates the raw scalogram, obtained by continuous wavelet transformation. The last three lines illustrate the effect of different normalisation techniques on the aspect of the scalograms. ARIMA and Diff are two techniques for pre-whitening the temporal signal. ZHO is the normalisation technique applied directly on the scalogram as disclosed before.

A possibility considered by the Inventors would have been to supply these scalograms directly to a neural network for identification. However, the scalograms themselves might suffer from a lack of information under real conditions, with fool (visual) “patterns” resulting from non-pathological physiological variations, of artefactual origin or not. For this reason, according to the present disclosure, the control of the candidate event of interest by calculation of a given number of characteristics thereof is preferred.

Once the scalograms have been created, they are supplied to a neural network, for example a convolutional neural network (CNN), intended to “pre-select” the scalograms potentially related to anomalies of the electroencephalographic signals. To this end, the convolutional neural network has been trained beforehand. More particularly, the artificial neural network is trained on scalograms belonging to two categories: the first category comprises the events to be detected and the second category comprises events that are not those to be detected. The output layer of the network is a layer outputting a binary result, depending on whether the event belongs, or not, to the category of events to be detected: “1”, the event belongs to the category and “0” the event does not belong to the category.

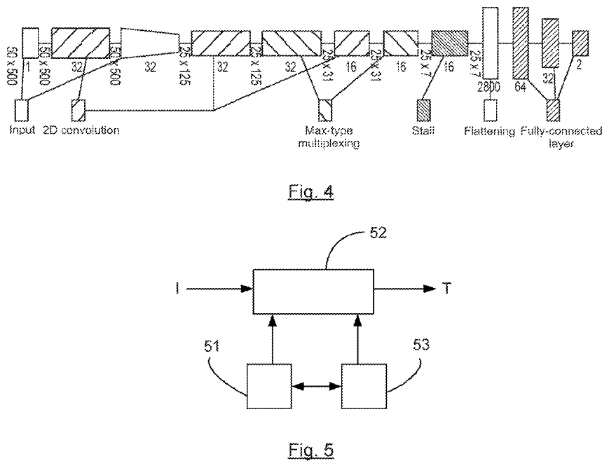

FIG. 4 illustrates an example of a convolutional network architecture as developed for the implementation of the present technique. The numbers and digits represent the configuration elements of the convolutional network for this implementation.

Then, the scalograms of the signal portions of the electroencephalograms to be studied are supplied to the network, so that the latter classifies them in either of the categories defined before.

The network performs a first classification (a first selection) on the basis of the scalograms supplied thereto. The signal portions having been used to generate the scalograms which have passed this pre-selection are then marked in the database (or in any other suitable data structure) and the second phase of the method is implemented: for each marked signal portion, a calculation of characteristics relating to this signal portion is performed.

In general, as indicated before, the considered signal portions, on which these characteristics are calculated, have been subjected to a spectral equalisation. The measured characteristics depend on the considered anomaly. Thus, the calculated characteristics are those that allow better differentiating the event with respect to the scalogram that has been subjected to pre-selection by the neural network, considering that the scalogram is in some way an imperfect graphical representation of the event, but a graphical representation carrying enough information useful for a pre-selection, to the extent that it is discriminating enough to eliminate most signal portions. Moreover, the scalogram carries overall information on the signal portion. What is more, the pre-selection carried out using the proposed method is performed more quickly and more efficiently than is the case if each portion had to undergo intensive and complete calculations of multiple characteristics. In other words, passing through a pre-selection based on a neural network allows expending less time and less energy to discriminate, even imperfectly, the signal portions than having to apply complex analytical processing on each signal portion. Thus, the developed technique allows being more efficient and more accurate than existing techniques in the processing of these large data sets.

Several techniques can be used to identify the remarkable portions of the filtered temporal signal: Hilbert envelope (by default), Hann envelope, sliding energy, moving average. These different measurements are more or less strongly correlated, thus the use of either one could have influence on the remainder of the operations, in particular depending on the events that are actively looked for.

Depending on the embodiments, the threshold used to locate the remarkable events from the envelope, the sliding energy or the moving average could vary between the 95th percentile and the 99th percentile. The higher the threshold, the more the number of false alarms decreases at the expense of a loss of true positives. The use of the 95th percentile allows being very cautious (limiting omission of true positives), the use of the 99th percentile allows being very strict and facilitating the interpretation of the results, in particular by reducing the time necessary to sort out false alarms.

Depending on the embodiments, the points of the envelope, the sliding energy or the moving average that exceed the threshold could be grouped when they are separated by less than 3 milliseconds, to form clusters.

Depending on the embodiments, the clusters of points may be kept if their total duration is within a duration that could vary from 6 to 8 ms. The lower the selected duration threshold, the more cautious we will be (the risk of rejecting true positives decreases). The higher the threshold, the more the probability of detecting false alarms will decrease. The threshold may also be set automatically, so as to adapt to the dominant frequency bands in the detected event (for example automatically set at 6.6 ms for a dominant event at 600 Hz, which corresponds to 4 oscillatory cycles, or 4 peaks, at this frequency).

Depending on the embodiments, several clusters may be grouped when the distance that separates them is within a duration that could vary from 2 to 8 ms. One objective of this strategy is to consider, as a unique event, an “interrupted” oscillation, i.e. undergoing a more or less significant transient loss of intensity on the filtered temporal signal, before regaining intensity.

Depending on the embodiments, the number of peaks contained in the temporal signal located “under” the envelope, the sliding energy or the moving average may vary between 2 and 6. The lower the threshold, the more cautious we will be (the risk of rejecting true positives decreases). The higher the threshold, the more the probability of detecting false alarms will be reduced.

Upon completion of the calculation of the characteristics (the number and the nature of which vary depending on the events looked for), the signal portions whose calculated characteristics are within prerequisite value ranges are marked again in the database and labelled as candidates for a complementary analysis. This complementary analysis, which is intended to determine whether or not the signal portions are actually representative of an event looked for, are processed afterwards either by a practitioner (or any other authorised operator), or analysed and processed again by an electronic device, for example implementing another neural network and/or any other suitable automated or semi-automated process.

### Description of a Specific Embodiment

In this embodiment, the implementation of the method proposed by the Inventors is specifically described in order to detect signal portions comprising one or more fast oscillation episodes (FR), within intracranial EEG signals obtained via intracranial probes (Macro/micro). In this implementation, long-term recordings (several tens of hours, and possibly several hundreds of hours) are provided. For example, processing one hour of signal over 70 channels consists in reviewing 504,000 signal portions (3,600 seconds×2 windows per second of signal×[70 channels]). Thus, the amount of data available for each patient is huge: when 200 hours of recording are provided, as this might happen in clinical conditions, approximately 100 million signal portions are processed by the technique developed by the Inventors. Since it is unconceivable to ask practitioners for a thorough FR detection study for each patient because the size of the data is colossal (because, unlike PEIs, FRs cannot be used in the clinical routine because they are strictly invisible to the naked eye at the commonly used scales), the described technique allows providing them with automated tools for detecting physio-pathological anomalies, in a first approach, which decreases by a 250 factor the number of signal portions to be analysed. In other words, a practitioner or another analysis device processes 250 times less data, and that being so with no cognitive bias. Thus, over the hundred million initial recordings, only four hundred thousand are “thoroughly” processed to determine whether or not they could be categorised as FR.

In this embodiment, the FRs are differentiated from the PEIs by their low intensity, their shorter duration, their high-frequency component and their periodic activity (3 or 4 periods at least). Since FRs are very short and local events, they are almost impossible to detect with the naked eye using conventional tools for visualising raw curves. To observe them, different types of simultaneous displays on the screen should be used: the raw signal, the filtered signal between 200 and 600 Hz and possibly a scalogram, which rapidly fills the space available on the screen. This is all the more as the displayed temporal portions are in the range of 400 to 600 ms. Only a few recording channels (3 to 6) can be visualised simultaneously, which makes the manual search for FRs very long and tedious to the user.

Thus, in this embodiment, the first step, implemented by the neural network, is based on the visual aspect of the normalised scalograms. To optimise the detection task, a sequential detection in the signal, by short sliding windows, is performed. When the scalogram is representative of an FR, it is retained as an event of interest. More particularly, the convolutional neural network (CNN) analyses the scalograms, which are produced for frequencies between 200 and 600 Hz, by 400 ms portions and a sliding window (the manner in which the scalograms are produced is described before). When an event is classified in the candidate FR category, the second step of the detection process is applied to reject false alarms.

The second step, in this FR case, consists of a temporal signal analysis. This step concerns a much lower number of signals than the first one, since it is carried out only provided that an event has passed the first filter implemented by the convolutional neural network. The analysis is then brought to the location of the filtered signal concerned by the distinctive characteristic of the scalogram, to verify the oscillatory characteristics of the FR candidate. Several elements are then controlled: the amplitude should exceed that of the background noise, the oscillation should contain at least 4 periods and its duration should exceed 6.7 ms (4 oscillations at 600 Hz). If an event meets all of the previous criteria, it is probably an FR and it is selected as such in a selection data structure created to this end.

More particularly, in this second step, for the case of FRs, the algorithm proceeds with this step through the quantification of the number of oscillations, the duration and the amplitude of the candidate event. To estimate these values, the Hilbert envelope of the signal is calculated over the entirety of the considered time window, namely generally 400 ms. All of the extreme points of the envelope, i.e. beyond the 97.5th percentile, are identified and compared. If several extreme points succeed one another without any interruption longer than 2 ms, they are considered to belong to the same group. If a group of points meeting these criteria exceeds 6 ms, it is considered as likely to include an FR under its envelope. If the average amplitude of the points of the considered envelope is at least twice as high as the amplitude of all of the other points of the envelope, the second quantitative criterion is validated.

Finally, a peak detection function is applied to the signal portion contained under the envelope portion of interest. If the event consists of at least 4 positive peaks, the last criterion is validated. The event will be categorised as a true positive. If one of these criteria is not met, the event will be categorised as a false positive. Following this step, the events classified in the “true positive” category are kept and selected for subsequent use (by a human operator or by a specialised complementary device).

In other words, in this second step, it is assumed that an FR is necessarily visible on the scalogram representing it, but the scalogram alone is not enough to ensure that a trace, even though it looks like it, is absolutely an FR. This is why other indices should be added to the decision-making. Thus, it is proceeded with this verification step by measuring the number of oscillations of the candidate FR and its amplitude, to eliminate false alarms. The number of oscillations (N_{osc}) is estimated using a peak detection function. The amplitude criterion is assessed from the Hilbert envelope, by calculating an amplitude Z-score (Zₑₙᵥ) between the average value of a portion of interest (ψ) and that of all the rest of the envelope (4). The values belonging to χ should meet two criteria: be located beyond the 97.5^{th} percentile of the envelope and be included in a group of points separated by less than 2 ms and the total duration of which (of the group of points) should exceed a duration that we shall call Dₘᵢₙ. Knowing that Nosc should be at least 4, Dₘᵢₙ can be calculated using the spectral mode (λ) of the oscillation as follows: Dmin=(4)/(λ*1000), namely 6.7 ms for a λ at 600 Hz and 20 ms for a λ at 200 Hz, for example. The Equation 1 expresses the calculation of the amplitude Z-score.

$${Z}_{e n ν} = \frac{\frac{1}{n} {Σ}_{i = 1}^{n} {χ}_{i} - \frac{1}{n} {Σ}_{i = 1}^{n} {ψ}_{i}}{{Σ}_{i = 1}^{n} \frac{{( {ψ}_{i} - \overline{ψ} )}^{2}}{n - 1}}$$

Once these characteristics are analysed and measured, it is considered that a candidate signal portion that meets all of the criteria is probably an FR. Hence, it is classified as such in the database for subsequent assessment (by an operator or another electronic device dedicated to this final assessment).

In addition, the described technique allows using the SEEG micro signal, and detecting the FRs recorded at this scale in order to better map the epileptic zone. Although this scale criterion (micro) adds an additional difficulty to a detection problem that is already extremely complex with the prior techniques, the technique of the invention allows processing these data obtained using the micro electrodes without adding more workload on the practitioner.

### Other Features and Advantages

Referring to FIG. 5, an electronic device for processing electroencephalographic data from a set of electroencephalographic recordings is described.

For example, the electronic data processing device comprises a memory 51 comprising for example a buffer memory, a general processor 52, equipped for example with a microprocessor, and controlled by a computer program 53, and/or a secure memory 54, a secure processor 55, controlled by a computer program 56, these processing units implementing methods for processing electroencephalographic data as described before to perform pre-detection of events of interest.

Upon initialisation, the code instructions of the computer program 56 are, for example, loaded into a memory before being executed by the secure processor 55. The processor 55 receives as input at least one electroencephalographic recording. The secure processor 55 implements the steps of the method, in particular to obtain a data structure in which some portions of electroencephalographic recordings are tagged according to the instructions of the computer program 56 to obtain a set of electroencephalographic signals that could be made available to a practitioner or to a computer for complementary visualisation or processing. For this purpose, besides the memory 54, the electronic device for processing electroencephalographic data comprises communication means, such as network communication modules, data transmission means and data transmission circuits between the various components of the electronic data processing device.

Moreover, the electronic device for processing electroencephalographic data (or the device implementing the described techniques) is provided with all the means necessary for the implementation of the previously-described methods, embodiments and variants.

Complementarily, the electronic device for processing electroencephalographic data comprises at least one data processing unit, a recording unit and at least one communication interface with a telecommunication network. In this specific embodiment, such a device comprises:
these means for extracting, from a set of starting scalograms, via an artificial neural network [trained, configured] beforehand, a set of candidate scalograms (Esc);
   For at least some candidate scalograms (Sc) of the set of candidate scalograms (Esc):
these means for calculating a plurality of characteristics of the electroencephalographic signal portion (So) corresponding to the candidate scalogram (Sc); and
these means for selecting the electroencephalographic signal portion (So) of the candidate scalogram (Sc) within an electroencephalographic signal selection data structure, said means being activated when the plurality of characteristics of the electroencephalographic signal portion (So) of the candidate scalogram (Sc) are located within prerequisite value ranges.

These means may be general means or dedicated means. For example, via a neural network, the extraction means may be in the form of a dedicated and architectured calculation unit for the implementation of artificial intelligence treatments.

1. A data selection method comprising:
selecting data derived from an electroencephalogram (EEG), said data being in the form of a set of starting scalograms, each scalogram of the set of starting scalograms being calculated from a portion of an electroencephalographic signal acquired beforehand, wherein the selecting is implemented by an electronic device and comprises:
extracting, from the set of starting scalograms, via the artificial neural network, a set of candidate scalograms;
for at least some candidate scalograms of the set of candidate scalograms:
calculating a plurality of characteristics of the electroencephalographic signal portion corresponding to the candidate scalogram; and
in response to the plurality of characteristics of the electroencephalographic signal portion of the candidate scalogram being within prerequisite value ranges, selecting the electroencephalographic signal portion of the candidate scalogram within an electroencephalographic signal selection data structure.

2. The data selection method according to claim 1, wherein said artificial neural network is a convolutional neural network.

3. The data selection method according to claim 1, wherein said artificial neural network is configured to detect “fast ripple” type fast oscillations within the starting scalograms.

4. The data selection method according to claim 1, wherein the characteristics of the electroencephalographic signal portion that are calculated belong to the group consisting of: duration, signal-to-noise ratio, a number of oscillations that compose the electroencephalographic signal portion, an amplitude of these oscillations, a shape of the oscillations.

5. The data selection method according to claim 1, wherein the method further comprises calculating each scalogram of the set of starting scalograms from a portion of the electroencephalographic signal by:
segmenting the electroencephalographic signal, according to a predetermined segmentation duration, outputting a plurality of electroencephalographic signal portions;
spectral equalisation of each electroencephalographic signal portion, outputting a plurality of equalised electroencephalographic signal portions;
calculating, from each equalised electroencephalographic signal portion, a scalogram using a wavelet transform.

6. The data selection method according to claim 5, wherein the calculation of the scalograms of the set of starting scalograms further comprises, for each scalogram obtained using a wavelet transform, normalising the scalogram.

7. The data selection method according to claim 1, wherein calculating the plurality of characteristics within the electroencephalographic signal portion corresponding to the candidate scalogram comprises:
calculating a Hilbert envelope of the electroencephalographic signal portion;
selecting, within the Hilbert envelope, a set of points located beyond the 97.5th percentile, called an extrema set;
selecting the set of extreme points following one another, with no interruption greater than 2 ms and a total duration of which is at least equal to 6 ms; and
calculating an amplitude and a number of positive peaks of the signal over the set of previously selected points.

8. The data selection method according to claim 7, wherein selecting the electroencephalographic signal portion of the candidate scalogram within the electroencephalographic signal selection data structure occurs when the average amplitude of the points of the set of previously selected points is at least twice as high as the amplitude of all of the other points of the Hilbert envelope and when at least four positive peaks are present on all of the previously selected points.

9. A device comprising:
at least one processor;
at least one non-transitory computer readable medium comprising instructions stored thereon which when executed by the at least one processor configure the device to select data derived from an electroencephalogram (EEG), said data being in the form of a set of starting scalograms, each scalogram of the set of starting scalograms being calculated from a portion of an electroencephalographic signal acquired beforehand, said selecting comprising:
extracting, from the set of starting scalograms, via an artificial neural network, a set of candidate scalograms (Esc);
for at least some candidate scalograms of the set of candidate scalograms:
calculating a plurality of characteristics of the electroencephalographic signal portion corresponding to the candidate scalogram; and
in response to the plurality of characteristics of the electroencephalographic signal portion of the candidate scalogram being within prerequisite value ranges, selecting the electroencephalographic signal portion of the candidate scalogram within an electroencephalographic signal selection data structure.

10. A non-transitory computer readable medium comprising a computer program product stored thereon comprising program code instructions for execution of a data selection method, when executed on a computer, wherein the data selection method comprises:
selecting data derived from an electroencephalogram (EEG), said data being in the form of a set of starting scalograms, each scalogram of the set of starting scalograms being calculated from a portion of an electroencephalographic signal acquired beforehand, the selecting comprising:
extracting, from the set of starting scalograms, via the artificial neural network, a set of candidate scalograms;
for at least some candidate scalograms of the set of candidate scalograms:
calculating a plurality of characteristics of the electroencephalographic signal portion corresponding to the candidate scalogram; and
in response to the plurality of characteristics of the electroencephalographic signal portion of the candidate scalogram being within prerequisite value ranges, selecting the electroencephalographic signal portion of the candidate scalogram within an electroencephalographic signal selection data structure.

11. The method according to claim 1, wherein the method further comprises:
acquiring the electroencephalographic signal from a patient using at least one electrode.
